(19) United States
(12) Patent Application Publication
(10) Pub. No.: US 2008/0299208 A1
(43) Pub. Date: Dec. 4, 2008

(54) Anisometric Particles In The Form Of Nanofibers/Mesofibers,Nanopipes, Nanocables/Mesocables, Nanobands/Mesobands, And The Curved Or Branched Variations Thereof

(76) Inventors: Joachim H. Wendorff, Nauheim (DE); Angreas Greiner, Amoneburg (DE); Thomas Schmehl, Giessen (DE); Tobias Gessler, Wettenberg (DE)

Correspondence Address: CLARK & BRODY, 1090 VERMONT AVENUE, NW, SUITE 250, WASHINGTON, DC, 20005, US

(21) Appl. No.: 11/666,653

(22) PCT Filed: Oct. 22, 2005
(86) PCT No.: PCT/DE2005/001897

§ 371 Date: Jul. 23, 2008

(30) Foreign Application Priority Data
Nov. 2, 2004 (DE) 10 2004 053 373.3

(51) Int. Cl. A61K 9/14; B06B 1/02; A61P 11/00; B29C 35/08

(52) U.S. Cl. 424/490; 264/442; 264/464; 264/478; 264/405; 264/454; 425/174.2; 425/174; 424/489

(57) ABSTRACT

The invention relates to novel anisometric mesoparticles and nanoparticles in the form of anisometric mesofibers/nanofibers, mesopipes/nanopipes, mesobands/nanobands, mesocables/nanocables, and the curved and branched or superimposed variations thereof as well as a novel method for the production thereof. The invention particularly relates to anisometric mesoparticles and nanoparticles which have an aerodynamic diameter <5 μm, the production thereof, loading thereof with active substances if the same cannot directly be utilized as an active substance, and the use thereof especially for producing medicaments against lung diseases or systemic diseases in humans and animals if the particles cannot directly be utilized as medicaments without carriers.

### TECHNICAL STATE OF THE ART

The invention at hand concerns anisometric particles, i.e. particles with a ratio of axes, which differs considerably from 1, in the form of meso- and/or nanofibers, meso-/nanotubes, meso-/nanoribbons, meso-/nanocables or/and their branched, or/and curved, or/and multi-coated variations.

In particular, the invention concerns for that purpose the provision of a method for the simple production of a multitude of anisometric particles with meso- or/and nanoscaled thickness with a defined, reproducible length, which avoids the disadvantages in the technical state of the art.

Furthermore, the invention concerns the provision of particles of pharmaceutically active agents or carrier particles, in particular for pharmaceutically active agents which avoid the known disadvantages of spherical aerosol particles and, in this, are preferably biodegradable or degradable under physiological conditions, in particular for the production of pharmaceuticals for application by inhalation for the treatment of lung diseases and/or systemic diseases of humans and animals.

The inhalation of pharmaceuticals—as just one area of application of the particles according to the present invention—is an established and widespread form of treatment of lung diseases, such as, for example, asthma and chronic obstructive lung diseases. Through application by inhalation, active agents, such as, for example, glucocorticoids, parasympatholytics, sympathomimetics, vasodilators, antibiotics etc., are suitable for being delivered to the site of the desired effect and side effects of oral or intravenous administration are avoided.

Furthermore, for the treatment of systemic diseases, active agents applied by inhalation are also suitable for being introduced through the large resorption surface and the extraordinarily thin gas-blood barrier of the lungs to the circulatory system and for being used for the treatment of various extrapulmonary diseases. For example, there has been success using insulin delivered as an aerosol for the treatment of diabetes, and, thus, avoid administration of insulin via syringe.

The following list, not to be understood as exhaustive, of diseases and pharmaceuticals thus comprises, amongst others, the following:

diseases:

asthma, COPD (chronic obstructive pulmonary diseases), pneumonia, mucoviscidosis, pulmonary hypertension, ARDS, lung cancer, lung metastases, fibrosing lung diseases, systemic diseases.

pharmaceuticals:

steroids, glucocorticoids, beta2 sympathomimetics, anticholinergics, secretolytics, methylxanthines, antiallergics, phosphodiesterase inhibitors, NO-donators, DNase, DNA, RNA, decoys, PAF receptor antagonists, leukotriene synthesis inhibitors and leukotriene receptor antagonists, prostaglandin D2 antagonists, IL-3/IL-5 antagonists, bradykinin antagonists, natriuretic peptides, insulin, proteins, opiates, antibiotics, antimycotics, virostatics, prostanoids, heparin, urokinase, elastase, hormones, cytostatics, immunosuppressants, natural or synthetic surfactant or its components, antioxidants, vitamins, and nicotine.

Until now, nebulizers or dosing aerosols, which consistently create nearly spherical pharmaceutical particles in the range of micrometers, have been used in the delivery of pharmaceuticals by inhalation. Said particles are deposited in the respiratory tract by sedimentation (sinking of the particles in the gravity field), impaction (carrier deposition through a change in direction of the gas flow carrying the particles) and diffusion (Brownian movement of the particles). Depending on their diameter, density and hygroscopicity, a certain portion of the pharmaceutical particles deposit in the mouth/throat area, in the tracheobronchial area (main airways) and in the alveolar area (gas exchange region of the lungs). A large portion of the particles, however, is not deposited in the respiratory tract, but rather expelled with the exhaled air; said portion of the inhaled pharmaceutical is not available for therapeutic effect.

A further disadvantage of the conventional medicinal aerosol therapy consists in the difficulty that it is hardly possible to reach a certain region of the lungs in a targeted form and with a high deposition rate, in order to treat a disease localized there, with the particles used until now.

With use of conventional spherical particles, a high deposition fraction in the tracheobronchial area, in particular, cannot be achieved (citation: Persons et al., J. Appl. Physiol. 63(3): 1195-1207, 1987). For example, asthma pharmaceuticals should be delivered to the tracheobronchial area of the lungs in order to achieve its effect locally there. Conventional pharmaceutical aerosols in the treatment of asthma, however, are always deposited to a high percentage in the alveolar area of the lungs as well, thus not at the desired localization of the effect.

An additional disadvantage of conventional pharmaceutical aerosols consists in the low biological half-life period of many pharmaceuticals—due to the short effective period at the location of deposition, it is thus necessary to repeat inhalation often in order to achieve a locally constant level of active agents and, therefore, a therapeutic effect. Pharmacological formulations with a controlled release of active agents are suitable for reducing the number of required inhalations and, furthermore, side effects, as strong fluctuations in concentration with high concentration peaks immediately after application are avoided.

In the past, different pharmaceutical formulations were proposed which provide a controlled, slow release of the active agents from a carrier system.

Thus, DE 40 21 517 A1, e.g., proposes a formulation with controlled release of a peptidic active agent, preferably somatostatin, such as octreotide, e.g. in the form of pamoate salt, wherein the active agent is located in a polymer carrier, preferably a polylactide-co-glycolide, specifically a poly(lactide-co-glycolide) glucose. The formulation is preferably in the form of monolytic microparticles, suitable for parenteral application.

DE 697 11 626 A1 proposes aerodynamic lightweight particles (comprising mainly polymers, in particular, functionalized polyester graft copolymers—for delivery of relatively large particles to release pharmaceutical agents to the lungs (airways and alveolar zones), wherein a particle system for release to the lungs, which contains biologically degradable particles with a tap density of less than 0.4 g/cm³, was proposed, wherein at least 50% of the particles should have an average mass mean diameter between 5 μm and 30 μm. Such carrier systems are, however, also, for example, liposomes or further polymer particles. Application of such or similar formulations by inhalation is carried out mostly through nebulization of the particles available in aqueous solution. Furthermore, it is possible to provide formulations of this nature in the form of powders for application by inhalation. In all these cases, nearly spherical particles are present, which are subject to the aforementioned limitations regarding deposition in the respiratory tract. In no case was the use of anisometric particles in the form of meso- and/or nanofibers, meso-/nanotubes, meso-/nanoribbons, meso-/nanocables or their curved and/or branched variations proposed for the inhalation of pharmaceuticals.

The known technical state of the art for production of meso-nano-scaled fibers, tubes or ribbons or cables comprises, e.g., the following specifications:

In patent number DE 100 53 263 A1 (“Oriented mesotubular and nanotubular non-wovens”), the production of compact nanofibers by electrospinning is described (see also Fong, H., Reneker, D. H. in “Electrospinning and the formation of nanofibers” in “Structure formation of polymeric fibers”, ed. Salem, D. R.; Sussman, M. V., p. 225-246, Hanser 2000).

Compact nanofibers from polymers which are biodegradable or soluble under physiological conditions are also suitable for being produced by electrospinning. Said polymers are suitable for being loaded with active agents during or after the electrospinning process.

In patent number DE 101 33 393 A1, the production of meso- or nanotubes (TUFT process) is described (see Bognitzki, M.; Hou, H.; Ishaque, M.; Frese, T.; Hellwig, M.; Schwarte, C.; Schaper, A.; Wendorff, J. H.; Greiner, A. Adv. Mat. 2000, 12, 637 and Hou, H., Jun, Z., Reuning, A., Schaper, A., Wendorff, J. H., Greiner. A., Macromolecules 2002, 35, 2429-2431). These are suitable for being produced from, amongst others, polymers which are biodegradable or soluble under physiological conditions and for being loaded with active agents.

In patent number DE 102 10 626.6 (“Method for producing hollow fibres” of M. Steinhart, R. Wehrspohn, U. Gösele J. H. Wendorff, A. Greiner), the production of nanotubes is described (see M. Steinhart, J. H. Wendorff, A. Greiner, R. B. Wehrspohn, K. Nielsch, J. Schilling, J. Choi, U. Gösele, Science 296, 1997 (2002)), which are likewise suitable for being produced from polymers which are biodegradable or soluble under physiological conditions and suitable for being loaded.

In the article “Flat Polymer Ribbons and Other Shapes by Electrospinning” by S. Koombhongse, W. Liu and D. H. Reneker from the University of Akron, Ohio, USA, published in “Journal of Polymer Science: Part B: Polymer Physics, Vol. 39, 2598-2606 (2001)”, it is reported that, apart from “ribbons” of polymer fibers, other fibers with asymmetric rotational cross sections are also suitable for being produced. In particular, it is shown that branched fibers are suitable for being produced.

Since it has, so far, been deemed a success to produce structures with a thickness of nano- or/and micrometers in a more or less commercial form at all, wherein extrusion methods, such as electrospinning, are mostly used, there is no method known in the technical state of the art which specifies how the aforementioned, mostly unsuitable or uneconomical methods are suitable for being modified to enable the production of a multitude of anisometric meso- or/and nanoscaled particles with a defined, reproducible length in a simple method and manner.

Furthermore, until now, no meso- or nanoscaled particles in a form and with an aerodynamic diameter were provided which avoid the disadvantages of the known spherical carriers.

### Aim of the Invention

Thus, the aim of the invention is

1) to provide a method for the simple production of a multitude of anisometric particles with meso- or/and nanoscaled thickness and with defined, reproducible length, which avoids the disadvantages in the technical state of the art.

2) to provide carrier particles, in particular for pharmaceutically active agents or essentially pure active agent particles, which, apart from non-polymer parts of active agents, comprise only formulation excipients, such as, e.g., water, e.g. composed of pure pharmaceutically active agents, which avoid the aforementioned disadvantages of spherical aerosol particles and, hereby, are preferably biodegradable or degradable under physiological conditions.

Aim 1) is achieved by the subject matter of patent claim 1.

The aforementioned aim 2) is achieved by the subject matter of patent claim 12.

### EMBODIMENTS

The newest research results concerning aim 2) have surprisingly found that it is advantageous, for targeted deposition of active agents, in particular, in the—for the treatment of asthma and chronic obstructive pulmonary diseases—important tracheobronchial area, as well as for increased deposition (in comparison to spherical particles) in the alveolar area, to provide anisometric active agent particle carriers, i.e. particles with a ratio of axes which differs considerably from 1, in the form of meso- and/or nanofibers, meso-/nanotubes, meso-/nanoribbons, meso-/nanocables or/and their branched, or/and curved, or/and multi-coated variations with or without nano/mesoscaled morphology of the surface, which comprise an aerodynamic diameter of less than or equal to 5 μm and, in the case of purely straight particles (single fibers, tubes, ribbons), a length between 10 and 500 μm.

An aerodynamic diameter of a particle is understood to be the diameter dₐ of a sphere with the density of 1 g/cm³, which comprises the same sedimentation rate as the particle.

The simplest form of an anisometric particle is, e.g., a straight meso-/nanotube or -fiber. For an aerodynamic diameter of 2,3,4,5 μm, the geometric diameters of such simple, linear, or purely straight particles at a length of 200 μm and e.g. (as assumed here) at a density of approx. 1 g/cm³ result in 1.0, 1.6, 2.20, 2.8 μm, i.e. 1000-2800 nm, see for this FIG. 1a, 1a′. The same is true for ribbons or cables, see FIG. 1b, 1b′ or crimped fibers and tubes.

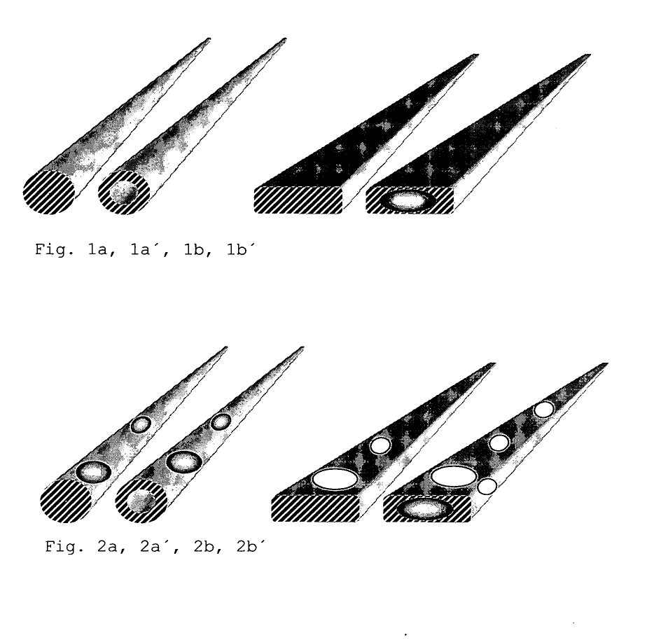

Straight meso-/nanotubes are suitable for being more complex forms according to the present invention, which comprise pores or pearl-shaped recesses as nano- or mesoscaled surface morphology, see FIG. 2a, 2a′, 2b, 2b′.

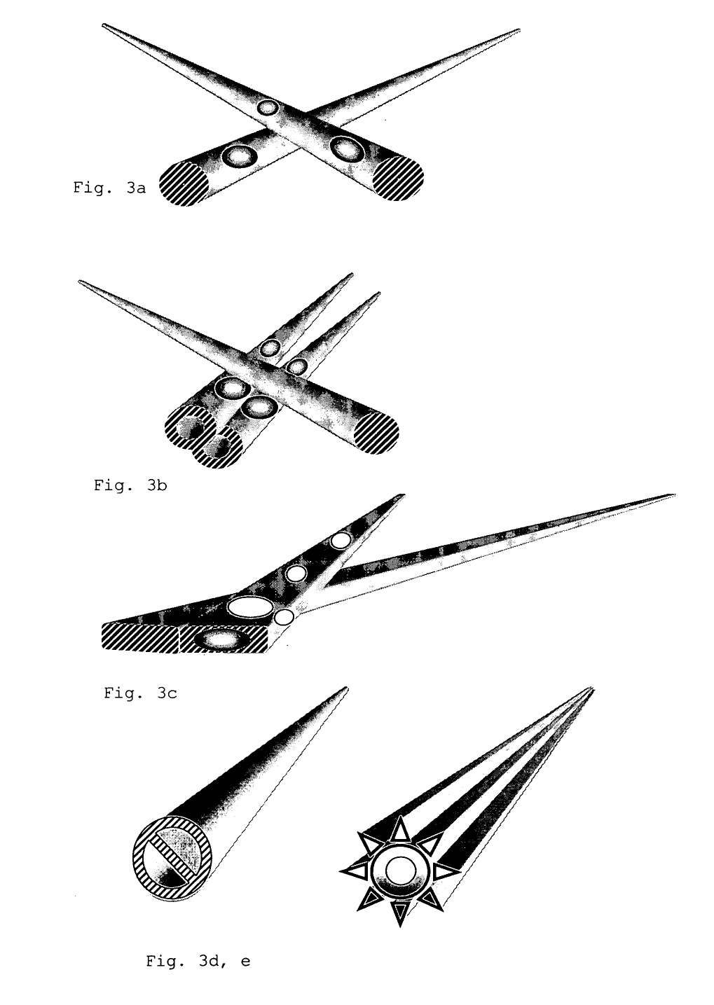

Even more complex forms according to the present invention are suitable for being, e.g., two tubes, which are superposed, i.e. connected to one another in the course of their length or at one end, or also multipods in the form of three or four tubes or ribbons connected to one another, which, e.g., are perpendicular to one another, see FIG. 3a, 3b, 3c, 3d,e,f,g,h.

The advantage of the invention regarding the solution of aim 2) exists in that the anisometric particles comprise a considerably increased deposition rate in the tracheobronchial area of the lungs in contrast to spherical particles of the same aerodynamic diameters. In this, the anisometric particles loaded with active agents are preferably available as a dry powder and are then preferably disaggregated by means of a suitable dry powder inhaler and made available for inhalation as an aerosol. A further advantage is that the release kinetic of the active agents contained is suitable for being controlled through form, dimensions and chemical composition.

For the solution of aim 1), according to the present invention, (provision of a simple method for the simple production of a multitude of anisometric particles to be provided with meso- or/and nanoscaled thickness and defined, reproducible length), the following basic procedural steps are proposed according to the present invention:

a) provision of starting materials, preferably, but not exclusively, in the form of polymers or/and other materials, or/and active agents or/and such mixtures, solutions, suspensions or emulsions (sol, gel, etc.) from one or several materials or/and polymers or/and active agents and, as far as necessary, solvents in a form which allows the production of anisometric fibers or/and ribbons with meso- or/and nanoscaled thickness and their branched forms through the methods of extrusion, melt blowing, solution blowing or electro- or co-electrospinning, which are known in principle.

b) production of anisometric fibers or/and ribbons or/and cables (single or multi-coated fibers or ribbons) with meso- or/and nanoscaled thickness and their branched forms through the methods of extrusion, melt blowing, solution blowing or electro- or co-electrospinning, which are known in principle.

c) shortening of the anisometric fibers or/and ribbons or/and cables (single or multi-coated fibers or ribbons) with meso- or/and nanoscaled thickness to the desired length by the influence of electromagnetic waves or sound waves.

Surprisingly, it was found that in the production of anisometric fibers or/and ribbons or/and cables with meso- or/and nanoscaled thickness, adjustment, which is able to be controlled and reproduced very well, of the desired length is possible. This is primarily due to the low thickness of the structures.

Thus, it was found that, due to the continuous feed movement during the methods of extrusion, melt blowing, solution blowing or electro- or co-electrospinning, which are known in principle, the influence of easily controllable, pulsed laser light of the most different wavelengths enables an effective transection of the anisometric fibers or/and ribbons or/and cables with meso- or/and nanoscaled thickness.

Surprisingly, this was also found for shortening through the use of ultra- or hypersound waves, as shown further below.

A particularly preferred embodiment of the above method for the production of pharmaceuticals to be administered by inhalation proposes the following basic steps:

a) provision of polymers or/and active agents and/or such mixtures, solutions, suspensions or emulsions (sol, gel, etc.), which are biodegradable or degradable under physiological conditions, from one or several pharmaceutically active agents, polymers and, as far as necessary, solvents, in a form which allows the production of anisometric particles by the methods of extrusion, melt blowing, solution blowing or electrospinning, which are known in principle,

b) production of active agent carriers or particles containing the agents prepared under a), which are biodegradable or degradable under physiological conditions, in the form of anisometric particles by the methods of extrusion, melt blowing, solution blowing or electrospinning, which are known in principle,

c) shortening of the anisometric particles by the influence of electromagnetic waves or sound waves to dimensions which correspond to an aerodynamic diameter of less than or equal to 5 μm. This embodiment has the advantage that the loading of the carrier particles occurs directly in the process of production of said particles. Thus, retroactive loading is unnecessary.

A very particularly preferred embodiment of the above method is given in that only one active agent or mixtures of active agents, preferably pharmaceutically effective active agents and, e.g., water as formulation excipients are provided as starting materials. Thus, surprisingly, pharmaceutically active agents containing proteins, e.g., were suitable for being transformed directly to particles of the aerodynamic diameters according to the present invention through electrospinning.

In the following, the different variation possibilities of the above method for the creation of further embodiments are given.

### Loading of Active Agents:

The loading of active agents of the anisometric particles to be produced is suitable for being carried out in basically three ways:

1) Thus, the active agents are suitable for being introduced into the starting solution for the production of particles (as shown in the above embodiment).

2) Or, they are suitable for being subsequently introduced into the particles via diffusion, in the presence of expanding agents, such as, e.g., supercritical carbon dioxide. The filling degrees set in this is, according to usage and type of extender, between less than 1% and up to 50%.

3) Finally, the active agents are suitable for being introduced directly to the fibers by a co-electrospinning process.

I) Production of Anisometric Particles in the Form of Fibers, Tubes, Ribbons or Cables and Their Curved and/or Combined and Bound Variations (Multipods)

### Linear Fibers:

Nano- or mesofibers are known to be producible (not to be understood as an exhaustive listing) by extrusion, melt blowing, solution blowing or electrospinning.

The formation of fibers preferably occurs via electrospinning by means of a high electrical tension, attached between a nozzle and a counter electrode (see XX 1-10). The material to be spun is present hereby in the form of a melt or a solution and is transported through one or several nozzle/s. The electrical field leads to a deformation of the droplet leaving the nozzle via induced charges; a fine material flow is formed which is accelerated in the direction of the counter electrode. The material flow is deformed hereby, e.g., elongated by a process called whipping instability, reduced in diameter, subjected to wave-shaped deflections and, subsequently, deposited on a substrate. During the spin process, the solvent evaporates or the melt cools, respectively.

The fibers are deposited at a rate of several meters per second. Through adjustment of the concentration of the solution, of the attached field, of the temperature and the use of additives and further parameters, the diameters of the fibers achieved are suitable for being adjusted within a wide range. Fibers down to several nanometers are able to be thus produced.

Fibers from amorphous or partially crystalline polymers from block copolymers, from polymer alloys are suitable for being created in this way. Thus, e.g., nanofibers from such different natural and synthetic polymers were produced, such as polyamides, polycarbonate or polymethylmethacrylate, from polynorbornenes, from polyvinylidene fluoride, from cellulose, from polylactides. The precise adjustment of the control parameters for electrospinning, which are known, however, from literature (see above), is necessary for the respective material.

A large advantage of electrospinning is that water is also suitable for being used as a solvent, so that water-soluble polymers and water-soluble biological systems, or polymer-active agent systems are suitable for being spun. Examples are polyvinyl alcohol, polyvinylpyrrolidone, polyethylene oxide, polyacrylamide or polyacrylic acid.

### Complex Fibers via Electrospinning

Porous fibers are suitable for being produced through the use of solvent mixtures; fibers with pearl-shaped recesses are suitable for being produced through variation of the conductivity of the solutions. Branchings which lead to multipods are suitable for being produced through the adjustment of the attached tension (appearance of multijets) or through cutting of the deposited felt. Crimped fibers and tubes are available through a variation of the distance between both electrodes with utilization of, e.g., whipping instability.

### II) Tubes:

Nanotubes are known to be suitable for being produced, by the following methods (not exhaustive):

a) The TUFT process for the production of nano hollow fibers is based on the coating of, e.g., electrospun or polymer template fibers attained otherwise.

In the first step, said template fibers are produced with the desired diameter and surface structure, e.g., through electrospinning, possibly already doped with active agents (method, see above).

In the second step, the template fibers produced in this way are coated with one or several materials, which constitute the later walls of the nano hollow fibers.

In the third step, selective removal of the template fibers occurs, by, e.g., thermal degradation or by extraction with a solvent selective for the template fibers. As far as the active agents are already present in the template fibers, removal of the template fibers is also to be carried out selectively relative to the active agents, so that the active agents remain in the nano hollow fibers formed.

One method for the production of functionalized nano hollow fibers of this kind, via the TUFT process, was already described (X3). As far as no active agents are yet incorporated in the template fibers, active agents are suitable for being introduced after production of the nano hollow fibers, amongst others, according to the method for electrospun nanofibers described (see above, e.g. via diffusion or also permeation).

b) The WASTE method for the production of nano hollow fibers is based on the wetting of high energy surfaces (e.g. aluminum oxide, silicon) with a polymer melt or polymer solutions under formation of an extremely thin film (X4). The walls of the nanotubes are formed from said film through solidification of the polymer melt or through evaporation of the solvent. Nanotubes with aspect ratios up to 10,000 of a uniform length and diameter are suitable for being produced in a large number through selective removal of the template.

The loading of active agents is suitable for occurring according to one of the methods described above either during the actual production of the anisometric particles or retroactively (e.g. via diffusion or permeations). Nanotubes with recesses are available through the use of templates with variable pore diameters, and multipods are available if porous templates with branchings are used.

### III) Core-Shell Particles:

Core-shell particles are able to be produced through, amongst others, the shortening of the Tuft process, i.e. removal of the template fiber is not carried out. Furthermore, co-electrospinning leads to core-shell structures. It is further possible to produce fibers with a core phase from the active agent or from a solution of the active agent and a shell from a polymer or a polymer mixture by a co-electrospinning process.

Adjustment of the Aerodynamic Diameter of the Anisometric Particles

The shortening of the loaded or unloaded carrier and/or active agent particles, in particular, with the aim of adjusting the required aerodynamic diameter (in the case of producing carrier particles useful in inhalation) occurs through the continuous, periodic or aperiodic influence of one or several identical or different energy sources in the form of electromagnetic radiation or sound, in particular light or ultrasound.

Naturally, shortening is also suitable for occurring through cutting, wherein this must occur, however, at individual fibers, ribbons, cables, and leads to a deformation of the particles by the influence of heat on the cutting edge. With fibers and tubes arranged parallel, conventional cutting leads to linear formations. If felt-like arrangements are cut, then branched multipods are obtained. Shortening of anisometric particles in the form of linear arrangements or felt-like is also suitable for being achieved through milling processes in ball mills.

During the provision of polymers in the form of block copolymers, the production of those block copolymers, i.e. through the choice of the distances of a component which is degradable, e.g. by electromagnetic radiation, is already suitable for adjusting which length will comprise the anisometric particles (e.g. in the form of purely straight fibers or multipods in branched fibers, ribbons, cables) shortened by influence of the energy source/s.

In the case of shortening during the production process, i.e., e.g. after leaving from a nozzle and before deposition of the anisometric particles formed (in the form of fibers, ribbons, cables) on an underlayer located opposite the nozzle, the use of periodically or aperiodically interfering electromagnetic radiation, e.g. in the form of laser radiation, is preferred.

In the known electrospinning processes, the deposition rates are in the range of approx. 1-10 m/s. During the desired production of anisometric particles in purely straight form (i.e., e.g. fiber pieces) with an aerodynamic diameter of less than 5 μm, the process of shortening to 10-500 μm fiber length respectively is necessary. For shortening every 10 μm, at a deposition rate of 1 m/s, every 10̂−5 s the influence of e.g. laser light for the shortening of the spun fiber is required. This corresponds to a frequency of 100 kHz, which e.g. is able to be represented using laser systems, such as the Trumpf Lasercell TLC 46, through adjusting/release of the corresponding pulse rate of 100 kHz. For the production of purely straight, longer anisometric particles (up to 500 μm), correspondingly lower pulse rates are required.

Shortening through ultrasound is preferably to be carried out after the production of the anisometric particles in the form of parallel particles or felts therefrom.

In this, said particles are suitable for being deposited in a liquid, wherein said liquid preferably forms the counter electrode of an electrospinning apparatus to which an ultrasound source is then attached. The container for the uptake of the liquid and the anisometric objects, as well as the ultrasound source, are to be adjusted to one another in such a way that standing waves form in a pattern (net-shaped patterns are typical) in which so-called “nodes” (areas with positive interference or maximal amplitude of the waves) find themselves at a distance, which corresponds to the desired length of the multipods which are to be shortened.

Experiments for shortening via ultrasound have resulted in the following possibilities: with an ultrasound source with a frequency f1 of 10̂10 Hz (at the upper limit of the “typical ultrasound range” of 16 kHz up to over 10̂10 Hz), wavelengths in the range of λ1, W=149 nm resulted with water at room conditions, i.e. at a propagation velocity of cW=1490 m/s. Within the resulting standing waves, pressure nodes and antinodes are formed at an interval of λ1,W2 according to the known formula 1=m×λ1,W/2, wherein l is the measurement of the container in one dimension. Areas of high pressure or areas of negative pressure form at the pressure nodes or antinodes (formed through the known longitudinal waves formed in liquid). Areas with very high or absolutely no pressure gradients then are located in between. Altogether, it can be observed that fibers are separated at an interval of λ1,W. Through controlling the ultrasound frequency, i.e. at 10̂7 Hz, conditions are thus suitable for being adjusted at which fiber pieces (tube pieces, etc.) with a length of 150 μm are able to be produced. With hypersound waves (<10̂10 Hz) in liquids, correspondingly shorter fiber pieces (as approx. 150 nm) are suitable for being produced.

Through the corresponding design of the container, the patterns of the interferences—as known from sound trials with common club-moss spores—are suitable for being changed.

### Release of the Active Agent

Release of the active agents is suitable for being achieved, in principle, in different ways. Among the processes used in this are (not exhaustive)

a) the permeation of active agents which are bound to polymers through the coating of the core of the carrier (core of the cable) or through the uppermost coating of the carrier in the case of tubes, fibers or cables.

b) the biological degradation or degradation of the carriers or/and active agent particles under physiological conditions. Polylactides e.g. are polymers which are biologically degraded; polyethylene oxides are dissolved by water.

The permeation behavior of small molecules in polymer systems was examined very extensively (XX 11-16). The transport of material J (material flow) through a film of the thickness d is controlled by the concentration gradient (c₁-c₂)/d, as well as by the permeation coefficient P, for which: J=P×(c₁-c₂)/d is valid.

The permeation coefficient, in turn, is determined through the product of the diffusion coefficient D and the solubility coefficient S, so that: P=D×S is valid.

The observation is now that it is the diffusion coefficient, in particular, which has a strong influence on the permeation behavior. On one hand, it is very strongly dependent on the size, the molecular weight of the permeate. A typical example is, at otherwise identical conditions, the sinking of the diffusion coefficient in PVC from approx. 10⁻⁶ cm2/s to approx. 10⁻¹⁴ cm2/s at an increase of the molecular weight or of the van der Waals volume by a factor of 7.

The second determining factor on the diffusion coefficient is the temperature and, in this, the difference of the temperature at which diffusion is examined from the glass temperature, in particular. Further determining factors are the morphology of the polymer film, i.e. the crystallinity degree, and the interconnectivity of the amorphous areas.

The permeation coefficient, which is determined through the factors of solubility and diffusion coefficient, is suitable for being adjusted over a wide range, typically between 10⁻⁴ cm2/s and 10⁻¹⁶ cm2/s. In order to be able to set the release of the active agents even more exactly, the fibers are suitable for being provided with a wall material or several concentric wall materials, through deposition from the gas phase or also through deposition from a solution phase, in connection to the production of said fibers, e.g. by means of electrospinning according to the so-called TUFT process (XX17,XX18). Through the choice of wall materials and their thickness, control of the release kinetic occurs.

Among the polymers which are available for release via permeation are, in particular, polymers such as polyurethanes, natural polymers, such as e.g. biological polycarboxylic acid and/or polysulfonic acids and/or sulfated polysaccachrides, polyacrylic acids, sulfonated polystyrenes, polyactides, polyvinylpyrrolidones, polyglycosides, polyamides, polyvinyl alcohols, polyvinyl acetates, polyvinyl ethers, polyethers, polyesters, polyimines, polyoxanones, starch, modified or non-modified celluloses, poly(lactide-co-glycolide, polyanhydride, gelatines, albumin, starch.

An advantageous embodiment of the anisometric particles is able to be produced in such a way that the surfaces of the nano-/mesofibers are produced in a structured manner. The structuring is completed hereby in such a way, that the surface of the fibers receives pores, i.e. general recesses in any form, in which active agents are then suitable for being stored easily, or pearl-shaped recesses, which comprise the active agents, e.g. for the variation of the release profile, amongst others, in the form of a pulsed release of active agents.

IV) Production of the Particles with Structuring of the Surface:

### WAY 1

Here, on one hand, the use of a binary system of one polymer and one solvent is possible. In the case of very volatile solvents, electrospinning leads to a depletion of the solvent and, thus, under certain known conditions, to a phase separation, to formation of a certain phase morphology, which then subsequently leads to a corresponding structuring of the fibers. The regular porous structure is noteworthy. The pores mostly comprise an ellipsoidal cross-section, wherein these, in the direction of the fiber axis, are approx. 300 nm long and, perpendicular to that, 50 nm to 150 nm wide.

### WAY 2

The second method provides the use of ternary systems of polymer 1/polymer 2/solvent. During the formation of the fibers, a segregation of both polymers occurs, if they are incompatible. Fibers are formed with a binodal (dispersoid phase/matrix phase) or also co-continuous spinodal structure. Such composite fibers are already interesting on their own. If one of the two components is selectively removed, then fibers with specific surface structure in the form of pores, ridges or grooves, as well, result. This method is already described in DE 100 40 897 A1 (“Production of polymer fibres having nanoscale morphologies”), wherein porous fibers from polymeric materials are proposed, which comprise fibers with diameters from 20 to 4,000 nm and pores (for instance, for the uptake of active agents) in the form of channels reaching at least to the fiber core and/or through the fiber.

Said fibers are to be produced according to claim 7 of the aforementioned specification in such a way that a solution containing 5 to 20 wt. % of at least one polymer is spun in a very volatile organic solvent or solvent mixture by means of electrospinning in a field of more than 10̂5 V/m, wherein the resulting fiber comprises a diameter of 20 to 4,000 nm and pores in the form of channels extending at least to the fiber core and/or through the fiber. Hereby, surfaces of 100 up to 700 m²/g are able to be realized. According to a preferred embodiment of the subject matter of said specification (column 4, paragraphs [0028] and [0029]), fibers, which initially do not comprise channels, are also able to be produced by using two polymers (one water-insoluble and one water-soluble). Said channels appear, however, when the water-soluble polymers are dissolved from the pores associated with them by the influence of water. For more precise production conditions, refer to said specification.

### Release of Active Agent:

Composite fibers, which are produced according to the TUFT process or the coelectrospinning process described above, are also suitable for being used for release of the active agent. In this case, both the template fibers and the wall materials must be either biodegradable or soluble under physiological conditions. For that purpose, the use of the following polymer classes is proposed: polylactides e.g. are polymers, which are biologically degraded; polyethylene oxides are polymers, which are soluble under physiological conditions. Composite fibers of this type are of particular interest for the adjustment of specific release profiles of active agent or for the combination of active agents.

A further advantageous embodiment of the subject matter of the invention comprises such anisometric particles (fibers, tubes, ribbons, cables, and their curved or superposed forms) as carriers, which comprise an aerodynamic diameter <3 μm and, in the case of the purely straight embodiment (meso/nanotube, -fiber, -ribbon, -cable) a length of 20-200 μm.

A further advantageous embodiment of the subject matter of the invention comprises such anisometric particles (fibers, tubes, ribbons, cables, and their curved or superposed forms) as carriers, which are built from several coatings or walls, so that the release of active agent is able to be adjusted even more targetedly. These are, e.g. able to be applied in-situ through the method of co-electrospinning on a core fiber or through the known methods, e.g., the method of gas phase deposition or through the less complex method of immersion or spraying on the core of a fiber or a ribbon.

The following figures show concrete embodiments for the anisotropic particles according to the present invention.

It is shown in:

FIG. 1a, 1a′, 1b, 1b′: linear anisometric particles (fibers, elliptical tubes, ribbons, hollow ribbons)

FIG. 2a, 2a′, 2b, 2b′: more complex linear anisometric particles (fibers, tubes, ribbons, hollow ribbons) with pore- or pearl-shaped recesses according to FIG. 1a, a′,b, b′

FIG. 3a: bound, linear anisometric particles (multipods): fibers with pore- or pearl-shaped recesses according to FIG. 1,2

FIG. 3b: bound, linear anisometric particles (multipods): fiber and several tubes with pore-/pearl-shaped recesses

FIG. 3c: bound, linear anisometric particles (multipods): ribbons and hollow ribbons with pore-/pearl-shaped recesses

FIG. 3d, e: bound, linear anisometric particles (multipods): two half-circle-shaped hollow fibers bound with one another and e: hollow fiber with ribs (or parallel fibers)

FIG. 3f, g: bound, linear anisometric particles (multipods): f) four ribbons bound with one another and g) three hollow ribbons bound with one another

FIG. 3h: bound, linear anisometric particles: four hollow ribbons bound with one another at an angle of 90°

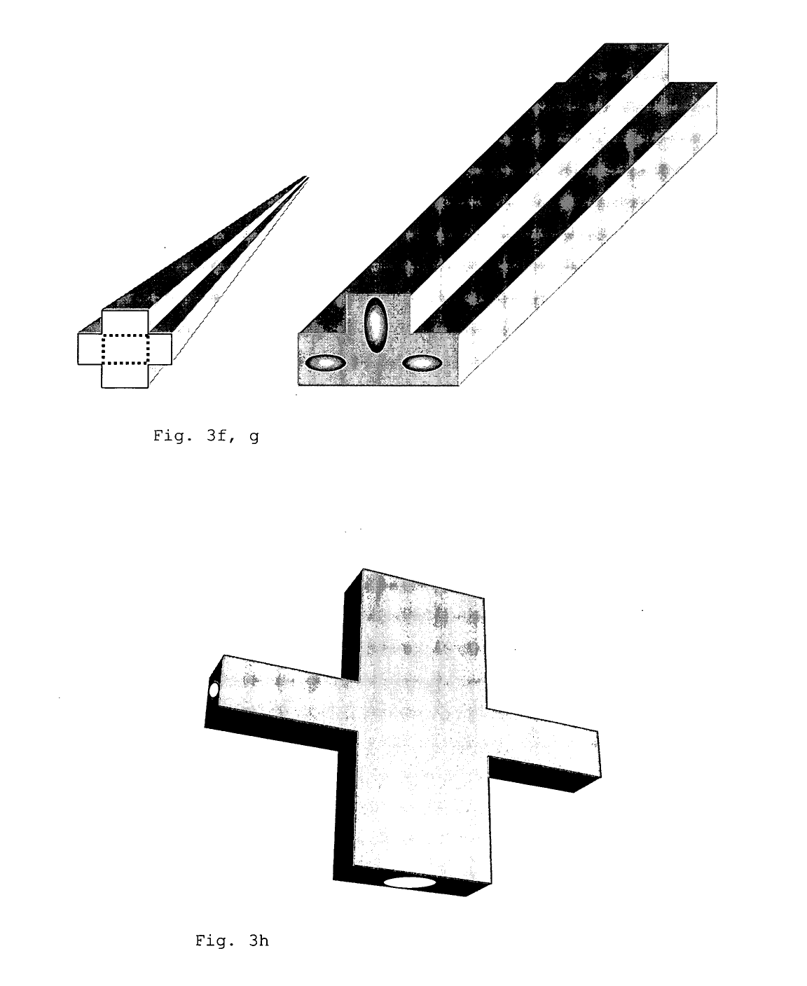

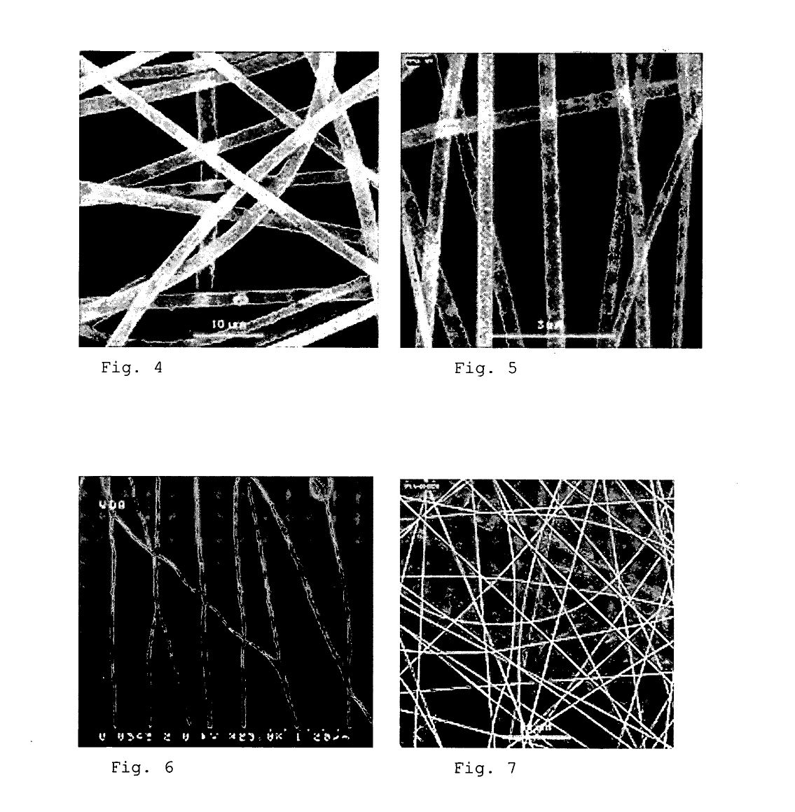

FIG. 4, 5, 6, 7 concrete embodiments for anisometric fibers with nanoscaled thickness.

FIG. 4 shows the result of electrospinning of polylactide (PLA, molecular weight Mw=630,000 g/mol, Mw/Mn=1.60) solvent dichloromethane. Concentration: 5 weight-% PLA. Distance between nozzle and planar counter electrode 15 cm. Tension 30 kV. Achieved diameters of the fibers 800-2400 nm.

FIG. 5 shows the results of electrospinning of polylactide (PLA, molecular weight Mw=630,000 g/mol, Mw/Mn=1.60) solvent dichloromethane. Concentration: 1 weight-% PLA.0.8 weight-% pyridinium formiate as additive. Distance between nozzle and planar counter electrode 15 cm. Tension 30 kV. Achieved diameters of the fibers 50-200 nm.

FIG. 6 shows the results of electrospinning of polylactide (PLA, molecular weight Mw=630,000 g/mol, Mw/Mn=1.60) solvent dichloromethane. Concentration: 1 weight-% PLA.0.8 weight-% pyridinium formiate as additive. Distance between nozzle and planar counter electrode 15 cm. Tension 30 kV. Achieved diameters of the fibers 10-70 nm.

FIG. 7 shows the result of electrospinning of polyvinyl acetate (PVA, molecular weight Mw=145,000 g/mol, Mw/Mn=1.60) solvent water. Concentration: 7 weight-% PLA.0.04 weight-% dodecyl sulfate as additive. Distance between nozzle and planar counter electrode 15 cm. Tension 30 kV. Achieved diameters of the fibers 100-200 nm.

### LITERATURE LIST

X1 Fong, H., Reneker, D. H. in “Electrospinning and the formation of nanofibers” in “Structure formation of polymeric fibers”, ed. Salem, D. R.; Sussman, M. V., p. 225-246, Hanser 2000
X2 Bognitzki, M.; Hou, H.; Ishaque, M.; Frese, T.; Hellwig, M.; Schwarte, C.; Schaper, A.; Wendorff, J. H.; Greiner, A. Adv. Mat. 2000, 12, 637.
X3 Hou, H., Jun, Z., Reuning, A., Schaper, A., Wendorff, J. H., Greiner. A., Macromolecules 2002, 35, 2429-2431
X4 M. Steinhart, J. H. Wendorff, A. Greiner, R. B. Wehrspohn*, K. Nielsch, J. Schilling, J. Choi, U. Gösele, Science 296, 1997 (2002)
XX2) Larrando, J.; Manley, R. S. J. J. Pol. Sci. Phys. Ed. 1981, 19, 909.
XX3) Doshi, J. N.; Shrinivasan, G.; Reneker, D. H. Polym. News 1995, 20, 206.
XX4)Jaeger, R.; Schonherr, H.; Vansco, G. J. Macromol. 1996, 29, 7634.
XX5) Reneker, D. H. ; Yarin, A. L. ; Fong, H. ; Koombhongse, S. J. Appl. Phys. 2000, 87, 9.
XX6) J. M. Deitzel, J. D. Kleinmeyer, J. H. Hirvonen, N. C. B. tan, Polymer 42, 8163 (2001)
XX7) Z. Chen, M. D. Forster, W. Zhou, H. Fong, D. H. Reneker, Macromol. 34, 6156 (2001)
XX8) Bognitzki, M.; Hou, H.; Ishaque, M.; Frese, T.; Hellwig, M.; Schwarte, C.; Schaper, A.; Wendorff, J. H.; Greiner, A. Adv. Mat. 2000, 12, 637.
XX10) Bognitzki, M.; Czado, W.; Frese, T.; Schaper, A.; Hellwig, M.; Steinhart, M.; Greiner A.; Wendorff, J. H. Adv. Mat. 2001, 13, 70.
XX11) J. Crank, G. S. Park, “Diffusion in Polymers”, Academic Press, N.Y., 1968
XX12) J. Comyn Ed., Polymer Permeability, Elesvier Appl. Sci. London 1986
XX13) H. B. Hopfenberg, V, Stannett in “The Physics of Glassy Polymers”, R. N. Haeward Ed. Applied Science Publ. London, 1973, p. 504
XX14) T. V. Naylor in “Comprehensive Polymer Science, S. G. Allen Ed., Pergamon Press, N.Y., 1989
XX15)F. Bueche, Physical Properties of Polymers, Interscience Publ. N.Y. (1962)
XX16) H. G. Elias, Makromoleküle, Hüthig und Wepf, Basel (1975)
XX17) Hou, H., Jun, Z., Reuning, A., Schaper, A., Wendorff, J. H., Greiner. A., Macromolecules 2002, 35, 2429-2431
XX18) Bognitzki M; Hou H Q; Ishaque M; Frese T; Hellwig M; Schwarte C; Schaper A; Wendorff J H; Greiner A., ADVANCED MATERIALS 2000, Vol 12, Iss 9, pp 637-640.

1. Method for the simple production of a multitude of anisometric particles with meso- or/and nanoscaled thickness and defined, reproducible length, characterized by the following basic procedural steps:
a) provision of starting materials, preferably, but not exclusively, in the form of polymers or other materials, such as active agents, in particular pharmaceutically active agents or/and such mixtures, solutions, suspensions or emulsions (sol, gel, etc.) from one or several of such materials or/and polymers and, as far as necessary, solvents in a form which allows the production of anisometric fibers or/and ribbons or/and cables with meso- or/and nanoscaled thickness and their branched or curved forms by the methods of extrusion, melt blowing, solution blowing or electro- or co-electrospinning, which are known in principle,
b) production of anisometric fibers or/and ribbons or/and cables (single or multi-coated fibers or ribbons) with meso- or/and nanoscaled thickness and their branched or curved forms by the methods of extrusion, melt blowing, solution blowing, or electro- or co-electrospinning, which are known in principle,
c) shortening of the anisometric fibers or/and ribbons or/and cables (single or multi-coated fibers or ribbons) with meso- or/and nanoscaled thickness and their branched or curved forms to the desired length by the influence of electromagnetic waves or sound waves.

2. Method according to claim 1, wherein in step a) at least two different starting materials or starting mixtures are provided, from which at least one of the two materials or mixtures is degradable, and which are transformed in step c) to a core- and shell-fiber or the corresponding ribbon-shaped components by co-electrospinning, wherein the degradable material or mixture forms the core part of the fiber, ribbon or cable and said material or mixture is degraded by the influence of the energy in step c) or subsequently through other known measures, such as chemical measures with the condition that a hollow, in particular, tube-shaped structure is obtained.

3. Method according to claim 1, wherein the starting materials comprise block copolymers with a degradable component, which are brought by step b) into a fiber form, ribbon form, cable form or their curved or associated variations, whereupon the degradable component is degraded by the influence of energy according to step c) in claim 1.

4. Method according to claim 1, wherein the electromagnetic radiation is applied as laser radiation, which, corresponding to the feed rate of the method chosen in claim 1, either step a) or b), is delivered in such a synchronized way that the desired length of the anisometric particles is achieved.

5. Method according to claim 1, wherein ultrasound or hypersound waves are used as sound waves, wherein the shortening of the fibers, ribbons, cables, tubes formed occurs particularly preferably through the formation of standing sound waves in air or/and gas or/and a liquid, wherein the desired length of the anisometric particles is able to be adjusted by the choice of the medium or from the choice of the medium and the choice of the container for the creation of the standing waves.

6. Method according to claim 5, wherein liquid is used for the creation of standing waves and the contact of the fibers, ribbons, cables or tubes with said liquid occurs through spraying with said liquid during step b), whereupon, for the purpose of shortening, standing waves are to be created by suitable sound sources and their arrangement within the liquid film formed on the fibers, ribbons etc. or by depositing the fibers, ribbons, cables, tubes on the surface of the liquid or in the liquid in a container, whereupon, for the purpose of shortening, standing waves are to be created on the surface or in the liquid of the container.

7. Method according to claim 6, wherein the liquid comprises components for the degradation of one of the starting materials or/and pharmaceutically or chemically or otherwise effective active agents, through which a tube is formed, or the nano-, mesoscaled structuring of the core or shell surface or the loading of particles with active agents occurs.

8. Method according to claim 6, wherein the liquid, in particular the surface of the liquid, in the case of depositing the fibers, ribbons, cables, tubes on or in the liquid, forms the counter electrode during the electro- or co-electrospinning.

9. Method according to claim 1, wherein the starting materials comprise polymers and/or such mixtures, solutions, suspensions or/and emulsions (sol, gel, etc.), which are biodegradable or degradable under physiological conditions, from one or several pharmaceutically active agents, polymers and, as far as necessary, solvents or active agents which are—apart from formulation excipients—pure, and the shortening of the anisometric particles occurs by the influence of electromagnetic waves or sound waves to dimensions which correspond to an aerodynamic diameter of the resulting particles of less than or equal to 5 μm.

10. Method according to claim 1, wherein the loaded active agent carriers are coated, e.g. for the production of cables, with a coat, which is permeable or diffusible for the active agents or active agent-polymer solutions or mixtures, during production, e.g. through spraying with a corresponding solution, or after production, e.g. through depositing on or in a corresponding solution.

11. Use of the method according to claim 1 for the production of active agent carrier particles or for the direct production of active agent particles which do not comprise polymer carriers, however formulation excipients if necessary, such as e.g. water before or/and after the process of production.

12. Anisometric carrier particles in a form which is loaded with active agent or/and free of active agent or/and pure active agent, wherein the carriers comprise parts of polymers which are biodegradable or—in the case of the form loaded with or free of active agent—degradable under physiological conditions in the bodies of humans or/and animals and the form of meso- and/or nanofibers, meso-/nanotubes, meso-/nanoribbons, meso-/nanocables or/and their branched, or/and curved, or/and hollow, or/and multi-coated variations, wherein the carriers comprise an aerodynamic diameter of less than or equal to 5 μm and, as long as available in straight form, e.g. as tubes or/and fibers or/and cables, a length of 10 to 500 μm.

13. Active agent carrier particles or active agent particles, produced according to claim 12, wherein the carriers or particles preferably comprise an aerodynamic diameter less than or equal to 3 μm.

14. Use of the method according to claim 1 for the production of a pharmaceutical for the treatment of lung diseases, in particular asthma and chronic obstructive pulmonary diseases of humans or/and animals.

15. Use of the method according to claim 1 for the production of a pharmaceutical for the treatment of systemic diseases of humans or/and animals.

16. Device for carrying out the method according to claim 1.

17. Device according to claim 16, wherein the device comprises pattern recognition means, i.e. means for the recognition of the particles created or the pre-structures.

18. Device according to claim 17, wherein the device comprises control means which are coupled with the pattern recognition means for the quality control of the length of the particles to be produced, preferably for the control of the feed rate of the fibers, ribbons, cables, tubes, e.g. in the case of electrospinning, through alteration of the pressure applied to the starting materials before leaving the nozzle.

19. Device according to claim 18, wherein the control means, in the case of shortening by the influence of pulsed electromagnetic radiation, control the pulse rate or the energy intensity of the applied pulses depending on the feed rate, as well.
